(19) United States
(12) Patent Application Publication
(10) Pub. No.: US 2024/0416112 A1
(43) Pub. Date: Dec. 19, 2024

(54) Systems And Method For Electrode Array Positioning

(71) Applicant: NOVOCURE GMBH, Root D4 (CH)

(72) Inventor: Christopher H. Scholl, West Chester, PA (US)

(21) Appl. No.: 18/743,707

(22) Filed: Jun. 14, 2024

Related U.S. Application Data
(60) Provisional application No. 63/508,072, filed on Jun. 14, 2023.

(51) Int. Cl. A61N 1/04; A47C 7/72; A61N 1/36

(52) U.S. Cl. CPC A61N 1/0476; A47C 7/72; A61N 1/36002; A61N 1/36014

(57) ABSTRACT

An apparatus for positioning and attaching an electrode array to a patient includes a chair having a seat portion and a backrest coupled to the seat portion, the backrest having a forward-facing side. The apparatus further includes at least one array attachment element on the forward-facing side of the backrest, the at least one array attachment element including an adhesive or a hook or loop material that is configured to releasably couple to the electrode array.

### CROSS-REFERENCE TO RELATED APPLICATION

This application claims priority to and the benefit of the filing date of U.S. Provisional Patent Application No. 63/508,072, filed Jun. 14, 2023, the entirety of which is hereby incorporated by reference herein.

### FIELD

This application relates to systems and methods for positioning and attaching electrode arrays to a patient.

### BACKGROUND

Tumor Treating Fields (TTFields) therapy is a proven approach for treating tumors using alternating electric fields at frequencies between 50 kHz and 1 MHz and, more commonly, between 100 kHz and 500 kHz. In current commercial systems, the alternating electric fields are induced by electrode assemblies (e.g., arrays of capacitively coupled electrodes, also called transducer arrays or electrode arrays) placed on opposite sides of a target region of the subject's body. When an AC voltage is applied between opposing electrode arrays, an AC current is coupled through the electrode arrays and into the subject's body.

Proper positioning of electrode arrays relative to each other and a target region (e.g., a tumor) can affect performance of treatment. However, proper placement can be difficult, particularly when the subject is placing the electrode arrays on himself/herself. Thus, this difficulty can diminish the independence of the subject, requiring the subject to have another person (helper) position the electrode arrays. Accordingly, a way to assist a subject with properly positioning one or more electrode arrays is desirable.

### SUMMARY

TTFields are approved for the treatment of glioblastoma multiforme (GBM), and may be delivered, for example, via the OPTUNE® system (Novocure Limited, St. Helier, Jersey), which includes transducer arrays placed on the patient's shaved head. More recently, TTFields therapy has been approved as a combination therapy with chemotherapy for malignant pleural mesothelioma (MPM), and may find use in treating tumors in other parts of the body. For applications targeting tumors in the torso, larger electrode arrays than currently used with the OPTUNE® system may be beneficial.

Disclosed herein, in at least one aspect, is an apparatus including a chair having a seat portion and a backrest coupled to the seat portion, the backrest having a forward-facing side. The apparatus further includes at least one array attachment element on the forward-facing side of the backrest, the at least one array attachment element including an adhesive or a hook or loop material that is configured to releasably couple to at least one electrode array.

Also disclosed herein, in another aspect, is a support structure that is configured to be secured to a backrest of a chair, the backrest having a forward-facing side. The support structure includes at least one array attachment element comprising an adhesive or a hook or loop material that is configured to releasably couple to an electrode array. The support structure is configured to be secured to the backrest of the chair so that the at least one array attachment element is on the forward-facing side of the backrest.

Methods of using the apparatus and support structure are also disclosed.

### BRIEF DESCRIPTION OF THE DRAWINGS

FIG. 1 is a perspective view of an exemplary apparatus for positioning and applying (attaching via adhesive) one or more electrode assemblies to a patient. The exemplary apparatus of FIG. 1 comprises a chair and a pair of array attachment elements coupled to the chair.

FIG. 2A is a perspective view of the apparatus of FIG. 1 with electrode arrays attached to the front face of the backrest of the chair via the array attachment elements. FIG. 2B is a schematic rear view of the backrest of FIG. 2A, showing flaps of a support structure overlapping and coupling to each other across the rear-facing side of the backrest to form a loop.

FIG. 3 is a perspective view of another exemplary apparatus as disclosed herein for positioning and attaching one or more electrode arrays to a patient.

FIG. 4 is a partial rear view of the apparatus of FIG. 3.

FIG. 5 is a partial front view of the apparatus of FIG. 3.

FIG. 6 is a rear view of an exemplary array attachment element.

FIG. 7 is a front view of the array attachment element of FIG. 6.

FIG. 8 is the front view of the array attachment element of FIG. 7 with an electrode array being applied thereto.

FIG. 9 is a schematic diagram of a system comprising an apparatus for applying an electrode array to skin of a user, as disclosed herein.

FIG. 10 is a perspective view of an exemplary apparatus for applying an electrode array to skin of a user, as disclosed herein.

FIG. 11 is a perspective view of a portion of the apparatus of FIG. 10.

FIG. 12 is a perspective view of an exemplary apparatus for applying an electrode array to skin of a user, as disclosed herein.

Various embodiments are described in detail below with reference to the accompanying drawings, wherein like reference numerals represent like elements.

### DETAILED DESCRIPTION

This application relates to positioning of electrode arrays that may be used, e.g., for delivering TTFields to a subject's body and treating one or more cancers or tumors located in the subject's body. Proper positioning of electrode arrays is more difficult when treating one or more cancers or tumor in the torso, particularly when optimum array placement may include placing one or more array on the patient's back. Disclosed herein are systems (apparatuses) and methods to facilitate the optimum placement of electrode arrays by a patient with or without the assistance of a medical professional or caregiver.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Headings are provided for convenience only and are not to be construed to limit the invention in any manner. Embodiments illustrated under any heading or in any portion of the disclosure may be combined with embodiments illustrated under the same or any other heading or other portion of the disclosure.

Any combination of the elements described herein in all possible variations thereof is encompassed by the invention unless otherwise indicated herein or otherwise clearly contradicted by context.

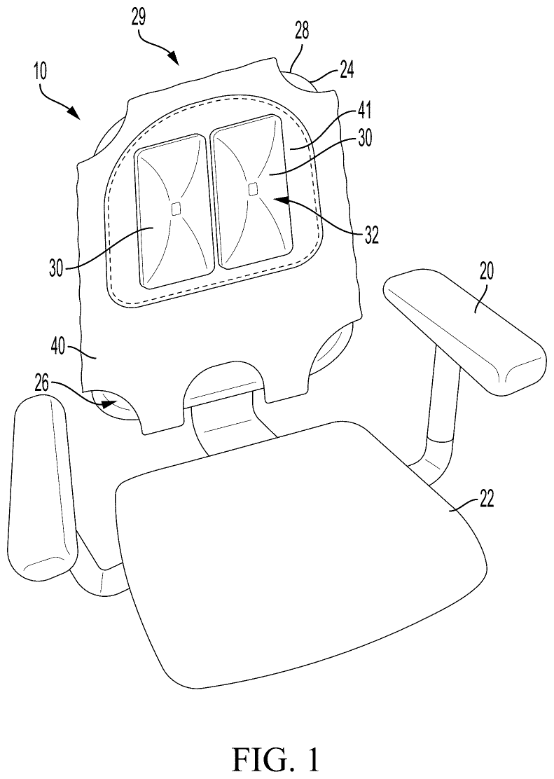

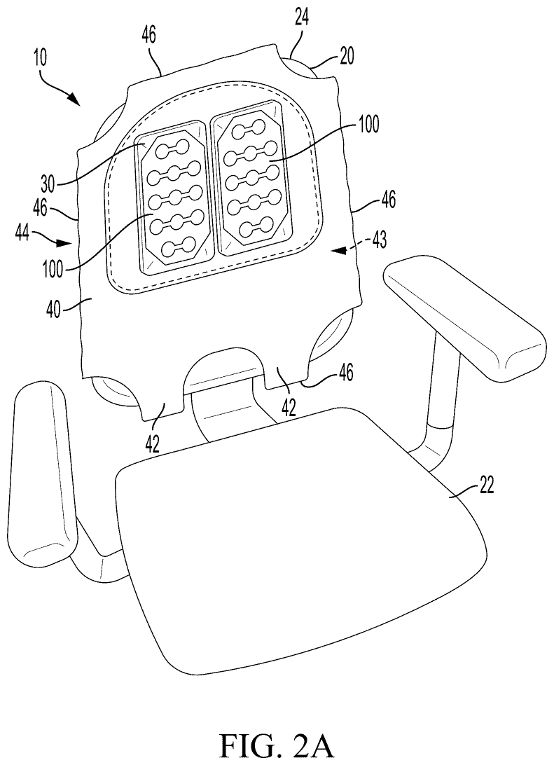

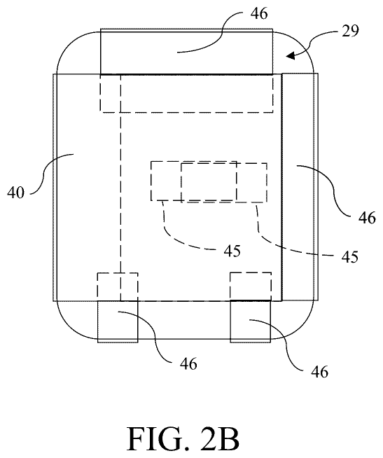

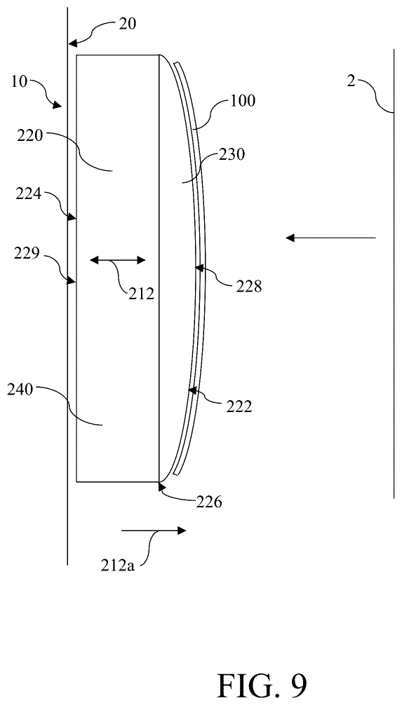

Disclosed herein, in various aspects, and with reference to FIGS. 1-2, is an apparatus 10 for positioning an electrode array 100 on a back 2 (FIG. 9) of a patient. The apparatus 10 can include a chair 20 having a seat portion 22 and a backrest 24 coupled to the seat portion, the backrest having a forward-facing side 26 and a rear-facing side 29. The apparatus 10 can further comprise at least one array attachment element 30 (also referred to herein as an “attachment element”) on the forward-facing side 26 of the backrest 24. The at least one array attachment element 30 can comprise an adhesive or a hook or loop material 32 that is configured to releasably couple to at least one electrode array 100. For example, in some aspects, the array attachment element 30 can comprise a pressure sensitive adhesive. Such pressure sensitive adhesives can be, for example, acrylic polymer adhesives. In other aspects, the array attachment element 30 can comprise a hook or loop material, an example of which is the VELCRO® brand of hook and loop fasteners from Velcro USA, Inc., Manchester, NH, USA. It is contemplated that the array attachment element 30 can comprise a hook material and the electrode array 100 can comprise a material, such as, for example, a nonwoven material that can serve as a loop material to which the hook material releasably attaches. In other aspects, the hook and loop materials can be positioned in the opposite sense, with the array attachment element 30 comprising a loop material and a surface of the electrode array 100 comprising a hook material. The adhesive or hook or loop material 32 can be selected to attach to the electrode array 100 sufficiently to hold the electrode array thereto under the weight of the electrode array, while weakly enough to release the electrode array therefrom when the electrode array is adhered to the patient. Accordingly, the adhesive or hook or loop material 32 can have a lower holding/release force than adhesive of the electrode array.

In some aspects, each array attachment element 30 can be embodied as the adhesive or the hook or loop material 32. In other aspects, and as further described herein with reference to some illustrated exemplary embodiments, the array attachment element(s) 30 can comprise one or more additional components that facilitate attachment of the electrode array 100 to the patient. For example, the array attachment element(s) 30 can be a compressible pad as further described herein. Still further, each array attachment element 30 can comprise means for attaching to the chair 20.

In some aspects, the apparatus 10 can comprise only one array attachment element 30. In other aspects, the apparatus 10 can comprise two, three, or more array attachment elements 30. For example, as illustrated, the apparatus 10 can have exactly two array attachment elements 30, with one configured for positioning a first electrode array 100 on a first location (e.g., a left side) of the patient and a second electrode array on a second location (e.g., a right side) of the patient.

In some aspects, the at least one array attachment element 30 on the forward-facing side 26 of the backrest 24 can be non-removably secured to the backrest 24 of the chair 20. In other aspects, and as described herein, the at least one array attachment element 30 can be removably coupled to the chair 20.

In some aspects, the apparatus 10 can comprise a support structure 40 that permits attachment of the one or more array attachment elements 30 to the backrest 24. Generally, the support structure 40 can comprise a structure for coupling to the backrest 24 of the chair and further provide a means for coupling the one or more array attachment elements 30 to the support structure 40. For example, the support structure 40 can comprise, an elastic sleeve or strap that extends over and/or around the backrest, thereby coupling the support structure to the backrest. The support structure 40 can further be removably or non-removably coupled to the at least one array attachment element 30. For example, in some aspects, the array attachment element(s) 30 can be stitched to or permanently adhered to the support structure 40. In other aspects, the array attachment element(s) 30 can be releasably coupled to the support structure 40 (e.g., via hook and loop material, one or more magnets, snap fasteners, or the like).

Referring to FIGS. 1-2, in exemplary aspects, the support structure 40 can be secured to the backrest 24. The support structure 40 can further couple to the at least one array attachment element 30 to thereby secure the at least one array attachment element to the backrest 24. The support structure 40 can couple to the chair 20 and position the at least one array attachment element 30 relative to the chair. For example, the support structure 40 can be secured to the backrest 24 of the chair 20 so that the at least one array attachment element 30 is on the forward-facing side 26 of the backrest. In this way, an off-the-shelf chair can be integrated into the apparatus 10. For example, in some aspects, the chair 20 can comprise an office chair. In further aspects, the chair 20 can be a household chair such as a dining room chair or any other suitable chair. In exemplary, optional aspects, the support structure 40 can comprise a fastener 41 (e.g., presenting hook or loop material, or one or more magnets or magnetic material) that is configured to permit attachment of the at least one array attachment element 30 thereto. In such cases, the apparatus 10 can comprise a support structure 40 that is secured to the backrest 24, and the support structure can comprise the fastener 41 and/or the at least one array attachment element 30. Optionally, the fastener 41 of the support structure 40 can be sized to permit positioning of the attachment element(s) 30 in different locations relative to the support structure 40. For example, the support structure 40 can comprise a sheet of hook material or loop material, and the array attachment element(s) can comprise the other of a hook material or loop material that is configured to couple to the sheet of hook material or loop material of the support structure 40. In other examples, the support structure 40 can comprise magnetic material (e.g., magnets or ferromagnetic material), and the array attachment element(s) 30 can comprise magnetic material (e.g., magnets or ferromagnetic material) that is configured to magnetically couple to the magnetic material of the support structure 40.

As illustrated in FIGS. 1-2B, the support structure 40 can comprise a strap 42 or a sleeve that defines an interior space 43 within which at least a portion of the backrest 24 can be received. In exemplary aspects, the support structure 40 can comprise an elastic material that resiliently stretches to receive at least a portion of the backrest. For example, the strap or sleeve that defines the interior space can resiliently expand so that the support structure 40 can be stretched over the backrest 24. In some aspects, at least one strap 42 of the support structure 40 can be adjustable. The support structure 40 can form a loop 44, with at least a portion of the at least one adjustable strap 42 defining the loop 44. The adjustable strap 42 can be configured to reduce an operative circumference of the loop 44 of the support structure to secure the support structure to the backrest. In this way, the adjustable strap(s) 42 can permit the support structure 40 to be adapted to differently sized backrests of chairs.

In some aspects, the support structure 40 can comprise at least one coupling element 45 that is configured to secure the support structure in a configuration in which the support structure forms a loop that surrounds at least a portion of the backrest. For example, in various aspects, the adjustable strap(s) 42 can comprise hook and loop material, snaps, one or more buckles, or any other suitable coupling element for securing the adjustable strap(s) 42 in a looped configuration. In some aspects, for example, the support structure 40 can comprise one flap or a plurality of flaps 46 that extend around and couple together on the back side of the backrest 24 to form one or more loops. For example, as illustrated, the flaps can fold around the backrest 24 to meet across a rear-facing side 29 so that the support structure 40 forms a loop 44 (or a plurality of loops).

The backrest 24 can have an upper surface 28 (FIG. 1). In some aspects, the support structure 40 can comprise at least one adjustable strap 42 that is configured to permit adjustment of a vertical position of the at least one array attachment element 30 relative to the upper surface 28 of the backrest. For example, the strap(s) shown as extending over the upper surface 28 of the backrest can be adjustable in length to adjust the height of the at least one array attachment element 30.

Although several embodiments of the support structure 40 are described herein as including an elastic sleeve or one or more strap, various other embodiments are contemplated. For example, in some aspects, the support structure 40 can comprise one or more hooks that extend over the upper surface 28 of the backrest 24. In other aspects, the support structure 40 can comprise one or more clamps that grip portions of the backrest 24, the seat portion 22, and/or one or more armrests of the chair 20. Generally, the support structure 40 can comprise any structure that couples to the chair and enables securing of the at least one array attachment element 30 along the backrest 24.

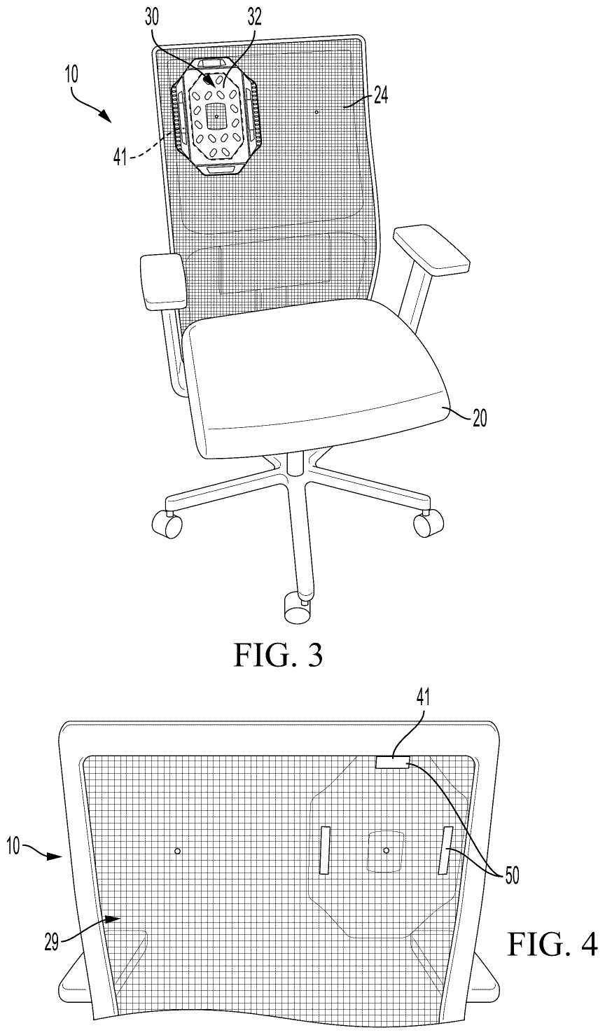

Referring to FIGS. 3-6, in some optional aspects, the support structure 40 can be omitted, and the electrode attachment element(s) 30 can be secured to the backrest 24 of the chair 20 without a support structure 40. For example, the apparatus 10 omitting the support structure can comprise one or more fastener 41 (e.g., optionally a magnet and/or magnetic material). In some exemplary aspects, as shown in FIGS. 3-5, the apparatus 10 can comprise at least one magnet (e.g., permanent magnet) 50 or at least one magnetic material 52. The at least one magnet 50 can be positioned on one of the forward-facing side 26 or the rear-facing side 29 of the backrest 24, and the at least one magnetic material 52 can be positioned on the other of the forward-facing side or the rear-facing side of the back rest so that the at least one magnet and the at least one magnetic material are coupled together through magnetic attraction through the backrest. Accordingly, the magnet 50 or magnetic material 52 positioned on the rear-facing side 29 of the backrest 24 can serve as the fastener 41. In exemplary aspects, the at least one magnetic material 52 can be a magnet. In other aspects, the at least one magnetic material 52 can be a material that is magnetized in the presence of a magnet (e.g., ferromagnetic material). In some optional aspects, the apparatus can comprise a plurality of magnets 50 (e.g., two magnets; or three magnets, as illustrated; or four magnets) and corresponding magnetic material elements 52. In exemplary aspects, the magnets 50 or magnetic material 52 can be positioned around a periphery of the array attachment element 30. In some optional aspects, the magnet(s) 50 or magnetic material 52 can be integral to the array attachment element 30. The attractive force between the magnet(s) 50 and magnetic material 52 can have sufficient strength to overcome a release force, as further described herein, between the at least one electrode array 100 and the adhesive or hook or loop material 32 of the array attachment element 30. For example, the magnet(s) 50 can be iron-based, or can be rare earth magnets.

The at least one array attachment element 30 can be configured to releasably couple to the at least one electrode array 100 with a release force that is less than an adhesive separation force of the at least one electrode array to the patient's skin. That is, each array attachment element 30 can be configured for use with an electrode array 100 that is designed to adhere to skin of a patient with a predetermined separation force, and each array attachment element 30 can be configured to couple to a respective electrode array 100 so that, when the patient moves away from the chair, the electrode array 100, having the greater separation force (separating the electrode array from the patient's skin) than the release force between the array attachment element 30 and the electrode array 100, remains adhered to the patient. The release force of the array attachment element 30 can be a function of, for example, its adhesive or of the hook (or loop) material used. In some optional aspects, the adhesive or the hook (or loop) material 32 of the array attachment element 30 can be selected based on the electrode array 100 to be used therewith.

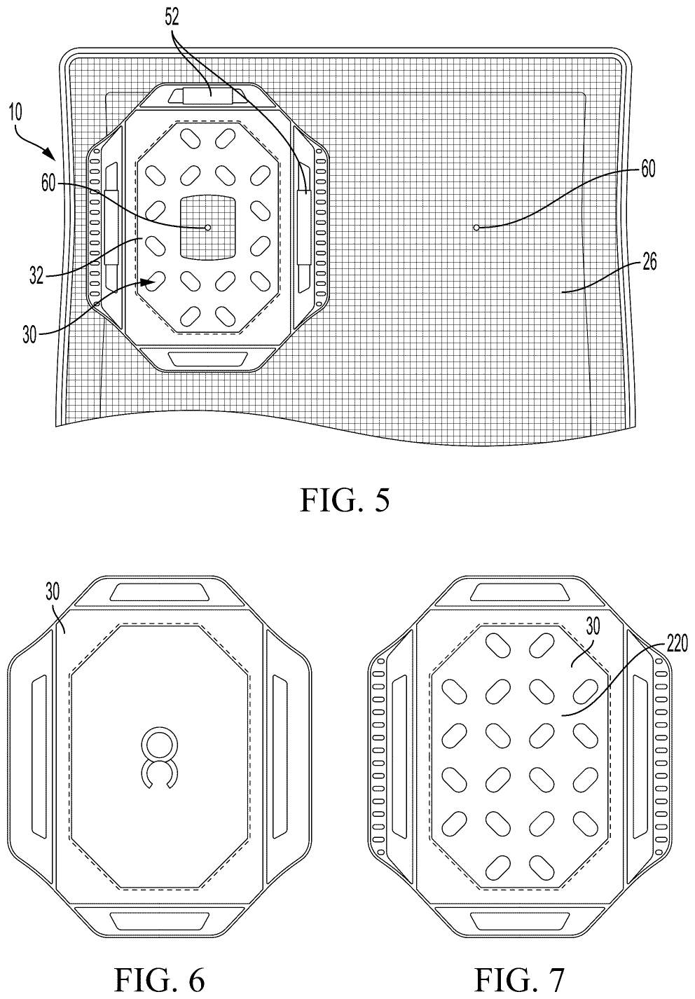

Referring to FIGS. 3-5, in some optional aspects, and as illustrated, the apparatus 10 can comprise a marking 60 (FIG. 5) for facilitating the positional attachment of each array attachment element 30 to the chair 20, for example, on the forward-facing side 26 of the backrest 24. In some aspects, each array attachment element 30 can comprise a window, and the array attachment element 30 can be positioned on the chair 20 so that the marking 60 is within (optionally, centered within) the window. In some exemplary aspects, a patient or medical professional can determine the location for the markings 60 (for example, on the backrest 24 of the chair, or on the support structure 40 or fastener 41) prior to attachment of the array attachment element(s) 30. That is, the chair can be marked for use with one or more array attachment elements 30. In exemplary aspects, said markings 60 can comprise, for example, stickers that adhere to the chair 20.

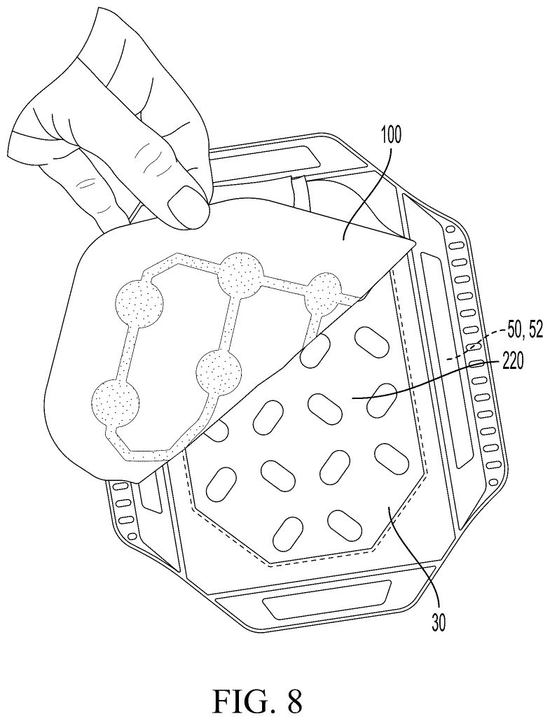

In exemplary aspects, the apparatus 10 can comprise at least one marking 60 indicating a location for attaching the electrode array 100 to the at least one array attachment element 30. Optionally, in these aspects, the at least one marking can indicate a position for placing a center of the electrode array 100. For example, a dot, an ‘X’, a ‘+’, or other marking can indicate the position for placing the center of the electrode array. In further or alternative aspects, the at least one marking 60 can include a marking indicating a position for placing an outer perimeter of the electrode array 100. For example, the at least one marking can trace an outer perimeter of the electrode array 100 when the electrode array is properly positioned. Optionally, as illustrated in FIG. 8, an outer edge of the array attachment element 30 can serve as the marking 60 that traces the outer perimeter of the electrode array.

FIG. 6 is a rear view of an exemplary array attachment element 30, and FIG. 7 is a front view of the array attachment element of FIG. 6. Referring to FIG. 7, in various aspects, the apparatus 10 can comprise an array attachment element 30 comprising at least one compressible pad 220 on the forward-facing side of the backrest 24. As further detailed below, the at least one compressible pad 220 can enhance adherence of the electrode array 100 to the skin of the patient. In exemplary aspects, the apparatus 10 can comprise two compressible pads 220. In some aspects, the compressible pad 220 can provide a protrusion for only a portion of the array attachment element 30. For example, the compressible pad 220 can extend across only an interior portion of the array attachment element. In this way, when using the array attachment element 30, an interior portion of the electrode array 100 that covers the compressible pad 220 can protrude relative to an outer periphery of the electrode array that extends beyond the compressible pad. Accordingly, the interior portion of the electrode array 100 can first contact the patient, and the outer periphery can then be pressed against the patient. This can inhibit air bubbles from being trapped between the electrode array 100 and the skin of the patient.

As discussed above, FIG. 8 illustrates the front view of an array attachment element 30 (which comprises a compressible pad 220) with an electrode array 100 being applied thereto. In FIG. 8, an outer edge of the array attachment element 30 can serve as the marking 60 that traces the outer perimeter of the electrode array.

Referring to FIGS. 9-12, in exemplary aspects, each compressible pad 220 can have a first (front-facing) side 222 and a second side 224 spaced relative to a first axis 212. The at least one array attachment element 30 (not shown in FIG. 9) can be secured to the at least one compressible pad 220 on the first side 222. The first side 222 can be configured to face the patient, and the second side 224 can be rear-facing. The front-facing side 222 can protrude (or otherwise extend) outwardly in a front-facing direction 212a along the first axis 212 to form an apex 228. That is, the apex 228 can correspond to the portion of the first side 222 located at an outermost axial position moving in a front-facing direction 212a along the first axis 212. In some aspects, the first side 222 can have an outer perimeter 226. Optionally, in these aspects, the apex 228 can be spaced from the outer perimeter 226. For example, in such aspects, the apex 228 can be substantially centrally located on the first (front-facing) side 222. In some aspects, the apex 228 can be defined as a region of the first side 222 having a maximal offset along the first axis 212 from any portion of the second side 224 of the compressible pad 220.

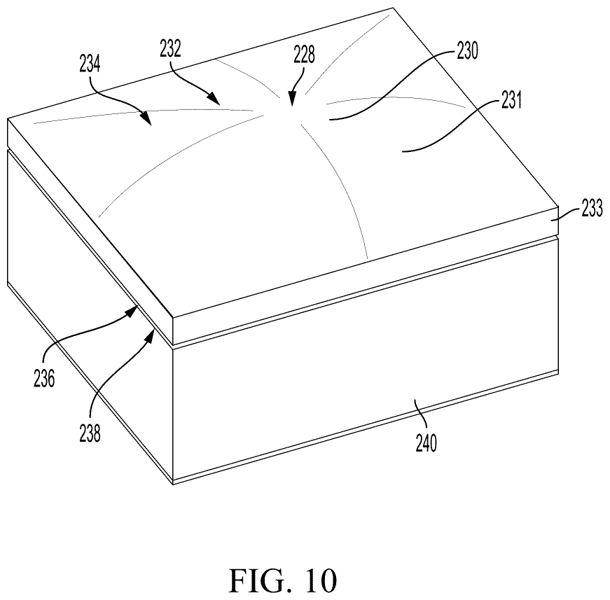

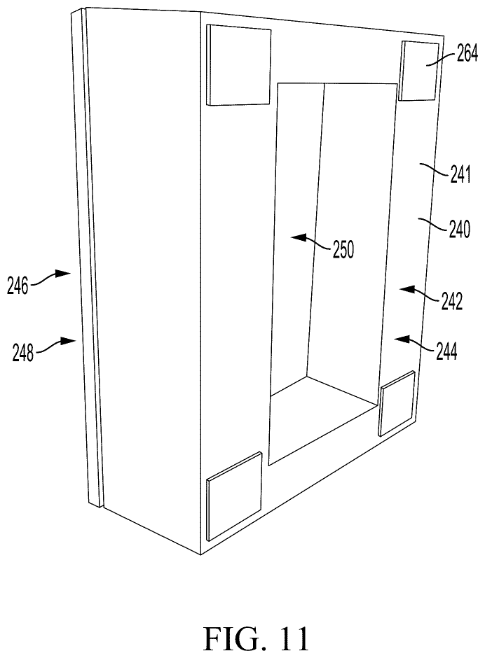

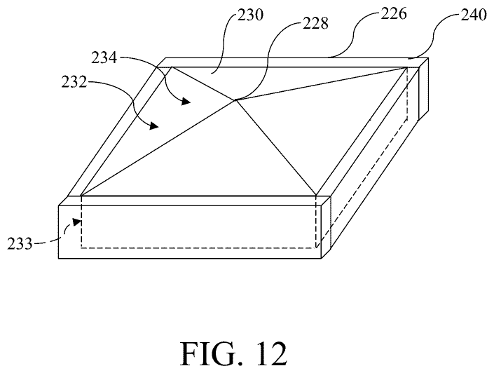

In some optional aspects, the compressible pad 220 illustrated in FIG. 9 can be formed from a single compressible material (e.g., foam) and, moreover, may comprise a single portion of the compressible material (e.g., foam). In other aspects, and referring to FIGS. 9-11, the compressible pad 220 can comprise a first portion 230 and a second portion 240. Referring to FIG. 10, the first portion 230 can have a first front-facing side 232 having a first front-facing surface 234 and a first back-facing side 236 having a first back-facing surface 238 (FIG. 10). The second portion 240 can have a second front-facing side 242 having a second front-facing surface 244 and a second back-facing side 246 having a second back-facing surface 248 (FIG. 11). The first front-facing side 232 (of the first portion) can protrude (or otherwise extend) outwardly in the front-facing direction 212a to form an apex 228. That is, a portion of the first front-facing side 232 can be spaced farthest from all parts of the second front-facing side 242 in the front-facing direction 212a along or parallel to the axis 212 to form the apex 228. It is contemplated that the single compressible material can form the first portion 230, and the second portion 240 can cooperate with the first portion 230 to form the compressible pad 220 illustrated in FIG. 9. In some exemplary aspects, it is contemplated that the first front-facing side 232 (of the first portion 230) as shown in FIG. 10 can be provided as an embodiment of the first side 222 (of the compressible pad) as shown in FIG. 9. The compressible pad 220 may comprise a first portion 230 and a second portion 240, which may be made of the same or different materials.

In some aspects, the second portion 240 of the compressible pad 220 can define a central void space 250 (FIG. 11). In some optional aspects, the central void space 250 can extend continuously through the second portion 240 of the compressible pad 220 from the second front-facing side 242 to the second rear-facing side 246. In these aspects, the central void space 250 can extend continually from the second front-facing surface 244 to the second back-facing surface 248. In other aspects, the central void space 250 can extend only partially through the second portion 240 of the compressible pad 220 from the second front-facing side 242 toward the second rear-facing side 246.

In some aspects, the first portion 230 of the compressible pad 220 can have a first indentation load deflection, and the second portion 240 of the compressible pad 220 can have a second indentation load deflection that is greater than the first indentation load deflection of the first portion. The indentation load deflection can be defined as a force (in the direction of a thickness of the material) required to compress a material sample (e.g., a 10 cm thick material sample) by 25% of its thickness. Accordingly, in some aspects, the second portion 240 of the compressible pad 220 can be more firm than the first portion 230. In the scenario of FIGS. 10-11, wherein the second portion 240 contains a central void space 250, the second portion 240 can apply a relatively large force against the peripheral edge of the electrode array 100 (as compared to the forces applied to other portions of the electrode array 100 by the apparatus 10). In some aspects, the second indentation load deflection can be at least 10% greater than the first indentation load deflection. In some aspects, the second indentation load deflection can be at least 25% greater than, at least 50% greater than, at least 75% greater than, at least 100% greater than, at least 150% greater than, or at least 200% greater than the first indentation load deflection.

In some aspects, at least a portion of the first back-facing side 236 of the first portion 230 fits within the central void space 250 defined by the second portion 240 so that at least 25% by volume of the first portion 230 is received within the central void space of the second portion. In further aspects, the first back-facing surface 238 can be positioned alongside the second back-facing surface 248 so that the first back-facing surface 238 and the second back-facing surface 248 cooperate to define a surface 229 (FIG. 9) of the second side 224 of the compressible pad 220.

In some aspects, the second side 224 of the compressible pad 220 can be planar or generally planar. In other aspects, the second side 224 of the compressible pad 220 can be concave. In other aspects, the second side 224 of the compressible pad 220 can be convex. In still other aspects, the second side 224 of the compressible pad 220 can be any other shape (e.g., wavy or otherwise uneven).

In some optional aspects, the compressible pad 220, in cross sections in planes parallel to the surface of the second side 224 of the compressible pad, can have a square, rectangular, or other polygon outer profile, or a generally square, or generally rectangular, or generally polygonal outer profile. In other aspects, the compressible pad 220, in cross sections in planes parallel to the surface of the second side 224 of the compressible pad, can have a circular, oval, or rounded profile. In still other aspects, the compressible pad 220, in cross sections in planes parallel to the surface of the second side of the compressible pad, can have any suitable profile. For example, optionally, the profile can match that of the shape of the electrode array 100 (such as the irregular octagonal shape shown in FIGS. 7 and 8).

In some optional aspects, all of the second portion 240 of the compressible pad 220 can be positioned peripherally around some or all of the first portion 230.

In some aspects, the compressible pad 220 can comprise foam. Optionally, in these aspects, the compressible pad 220 can comprise a first foam material 231 (FIG. 10) that defines the first portion 230 and a second foam material 241 (FIG. 11) that defines the second portion 240.

The first foam material 231 can have an outer peripheral surface 233. In some aspects, the second foam material 241 can be coupled to the outer peripheral surface 233 of the first foam material 231. For example, in some aspects, the first and second foam materials 231, 241 can be formed together via overmolding. In some aspects, the first and second foam materials 231, 241 can be adhered together (e.g., via adhesive or a fastener, such as hook and loop fastener). In other aspects, the first and second foam materials 231, 241 need not be coupled together. For example, in some aspects, the first foam material 231 can be removably received within the second foam material.

Referring to FIGS. 10-11, in some aspects, the first portion 230 of the compressible pad 220 can be removably coupled to the second portion 240. For example, in some aspects, the first portion 230 can be coupled to the second portion 240 via one or more hook and loop fastener 264. For example, the first portion 230 and the second portion 240 of the compressible pad 220 can be coupled together using one or more patches or strips of hook and loop material 264 on each of the first portion and the second portion. Optionally, in these aspects, one of a hook fastener or a loop fastener can be positioned on the second front-facing side 242 of the second portion 240 of the pad 220, and the other of the hook fastener or the loop fastener can be positioned on the first back-facing side 236 of the first portion 230 of the pad 220. In other aspects, any suitable fastening means, such as, for example, adhesive, can be used to couple the first portion 230 of the compressible pad 220 to the second portion 240.

In some optional aspects, the first front-facing surface 234 of the first front-facing side 232 of the first portion 230 of the compressible pad 220 can have a convex surface in a direction away from the first back-facing side 236. In some aspects, the first front-facing surface 234 of the first front-facing side 232 of the first portion 230 of the compressible pad 220 can have a surface shape comprising a partial spherical, ellipsoidal, paraboloidal or hyperboloidal shape. In other aspects, and as illustrated in FIG. 12, the first front-facing surface 234 of the first front-facing side 232 of the first portion 230 of the compressible pad 220 can comprise a plurality of planar or generally planar surfaces that meet at the apex 228. Optionally, the apex can be flattened rather than coming to a point. (FIG. 12 illustrates an embodiment wherein the first portion 230 fits within the central void space 250 of the second portion 240).

In some optional aspects, the compressible pad 220 can have a variable thickness. For example, the first side 222 of the compressible pad 220 can be spaced from the second side 224 along, or parallel to, the first axis 212 to define a thickness of the compressible pad, and the variable thickness can correspond to a distance between the first side and the second side along, or parallel to, the first axis 212. Optionally, in these aspects, the apex 228 can be positioned at the location of the greatest thickness of the compressible pad 220. Further, the thickness of the compressible pad can vary across the pad in dimensions perpendicular to the first axis 212.

The outer perimeter 226 bounds an area. In exemplary aspects, the apex can be located at, or approximately at, the centroid of the area bounded by the outer perimeter.

Referring to FIG. 9, a method of using the apparatus 10 can comprise releasably securing an electrode array 100 to the at least one array attachment element 30 (not shown in FIG. 9) via the adhesive or hook or loop material 32. For example, a respective electrode array 100 can be coupled to each array attachment element 30. Optionally, a release liner of the electrode array 100 can be removed to expose an adhesive of the electrode array 100. The patient can sit in the seat portion 22 of the chair 20 and can press his/her back 2 against the electrode array 100. For example, the patient can sit in the seat portion and then lean back to firmly press his/her back against the electrode array. In some aspects, the patient can hold his/her back against the electrode array for 10 seconds. In some aspects, the patient can hold his/her back against the electrode array for 20 seconds, or 30 seconds, or 60 seconds, or 5 minutes, or 10 minutes, or 20 minutes, or 30 minutes, or more. It is contemplated that the use of the chair can permit the patient to steadily hold his/her back against the electrode array for a sufficient time in order to permit sufficient or optimal adhesion of the electrode array. The patient can then lean forward to separate the electrode array 100 from the array attachment element 30. Because the electrode array 100 is placed on the array attachment element 30, which in turn is positioned on the backrest 24 of the chair 20 according to the optimum positioning for the electrode array on the patient's back, the array can be positioned and adhered to the patient's back in the optimum location for the TTFields treatment.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus comprising:
a chair having:
   a seat portion; and
   a backrest coupled to the seat portion, the backrest having a forward-facing side; and
at least one array attachment element on the forward-facing side of the backrest, the at least one array attachment element comprising an adhesive or a hook or loop material that is configured to releasably couple to at least one electrode array.

Aspect 2: The apparatus of aspect 1, further comprising:
at least one magnet; and
at least one magnetic material,
wherein the backrest has a rear-facing side,
wherein the at least one magnet is positioned on one of the forward-facing side or the rear-facing side of the backrest, wherein the at least one magnetic material is positioned on the other of the forward-facing side or the rear-facing side of the back rest, and wherein the at least one magnet and the at least one magnetic material are coupled together through magnetic attraction through the backrest, and further wherein the at least one array attachment element on the forward-facing side of the backrest is secured to the backrest of the chair by the at least one magnet and the at least one magnetic material.

Aspect 3: The apparatus of any one of the preceding aspects, wherein the at least one array attachment element is configured to releasably couple to the at least one electrode array with a release force that is less than an adhesive separation force of the at least one electrode array with skin of a patient.

Aspect 4: The apparatus of any one of the preceding aspects, further comprising at least one compressible pad on the forward-facing side of the backrest.

Aspect 5: The apparatus of any one of the preceding aspects, wherein the at least one array attachment element on the forward-facing side of the backrest comprises at least one compressible pad.

Aspect 6: The apparatus of any one of the preceding aspects, further comprising at least one compressible pad on the forward-facing side of the backrest, wherein each of the at least one compressible pad has a first front-facing side having a first front-facing surface which extends outwardly in the front-facing direction to form an apex.

Aspect 7: The apparatus of any one of the preceding aspects, further comprising at least one compressible pad on the forward-facing side of the backrest, wherein each of the at least one compressible pad has a first front-facing side having a first front-facing surface wherein the first front-facing surface has a convex surface in a front-facing direction.

Aspect 8: The apparatus of aspect 6 or aspect 7, wherein the first front-facing surface of the first front-facing side has a surface shape comprising a partial spherical, ellipsoidal, paraboloidal or hyperboloidal shape.

Aspect 9: The apparatus of any one of aspects 6-8, wherein the first front-facing side of the at least one compressible pad has an outer perimeter, and wherein the apex is located approximately at the centroid of the area bounded by the outer perimeter.

Aspect 10: The apparatus of any one of aspects 6-9, wherein the at least one compressible pad comprises foam.

Aspect 11: The apparatus of any one of the preceding aspects, wherein the apparatus comprises a support structure that is secured to the backrest, wherein the support structure is configured to couple to the at least one array attachment element to thereby couple the at least one array attachment element to the chair.

Aspect 12: The apparatus of aspect 11, wherein the support structure comprises an elastic material that resiliently stretches to receive at least a portion of the backrest.

Aspect 13: The apparatus of aspect 11 or aspect 12, wherein the support structure comprises at least one adjustable strap, wherein the support structure forms a loop comprising the at least one adjustable strap, wherein the adjustable strap is configured to reduce an operative circumference of the loop of the support structure to secure the support structure to the backrest.

Aspect 14: The apparatus of any one of aspects 11-13, wherein the support structure comprises at least one coupling element that is configured to secure the support structure in a configuration in which the support structure forms a loop that surrounds at least a portion of the backrest.

Aspect 15: The apparatus of any one of aspects 11-14, wherein the backrest has an upper surface, wherein the support structure comprises at least one adjustable strap that is configured to permit adjustment of a vertical position of the at least one array attachment element relative to the upper surface of the backrest.

Aspect 16: The apparatus of any one of aspects 11-15, wherein the backrest has a rear-facing side, wherein the support structure comprises:
at least one magnet; and
at least one magnetic material,
wherein the at least one magnet is positioned on one of the forward-facing side or the rear-facing side of the backrest, wherein the at least one magnetic material is positioned on the other of the forward-facing side or the rear-facing side of the back rest, and wherein the at least one magnet and the at least one magnetic material are coupled together through magnetic attraction through the backrest.

Aspect 16A: The apparatus of any one of the preceding aspects, further comprising at least one marking indicating a location for attaching the electrode array to the at least one array attachment element.

Aspect 16B: The apparatus of aspect 16A, wherein the at least one marking comprises a marking indicating a position for placing a center of the electrode array.

Aspect 16C: The apparatus of aspect 16A, wherein the at least one marking comprises a marking indicating a position for placing an outer perimeter of the electrode array.

Aspect 17: The apparatus of aspect 1, wherein the at least one array attachment element on the forward-facing side of the backrest is non-removably secured to the backrest of the chair.

Aspect 18: The apparatus of any one of the preceding aspects, further comprising:
at least one compressible pad having a first side that is configured to face toward a patient, and a second side that is configured to face away from the patient, wherein the at least one compressible pad comprises:
   a first portion; and
   a second portion,
wherein the first portion has a first front-facing side having a first front-facing surface and a first back-facing side having a first back-facing surface, wherein the first front-facing side is spaced from the first back-facing side in a front-facing direction, wherein the second portion has a second front-facing side having a second front-facing surface and a second back-facing side having a second back-facing surface, and wherein the first front-facing side is configured to face toward the patient,
wherein the first front-facing side extends outwardly in the front-facing direction to form an apex, and
wherein the at least one array attachment element is secured to the at least one compressible pad on the first side of the at least one compressible pad.

Aspect 19: The apparatus of aspect 18, wherein the second portion defines a central void space extending from the second front-facing surface toward the second back-facing surface, and wherein either:
the first back-facing surface of the first portion abuts the second front-facing surface of the second portion, or
at least a portion of the first back-facing side of the first portion is received within the central void space of the second portion.

Aspect 20: The apparatus of aspect 18 or 19, wherein the first portion has a first indentation load deflection, and wherein the second portion has a second indentation load deflection that is greater than the first indentation load deflection of the first portion.

Aspect 21: The apparatus of aspect 19 or aspect 20, wherein at least a portion of the first back-facing side of the first portion is received within the central void space of the second portion so that at least 25% by volume of the first portion is received within the central void space of the second portion, and wherein the first back-facing surface is positioned alongside the second back-facing surface such that the first back-facing surface and the second back-facing surface cooperate to define a surface of the second side of the at least one compressible pad.

Aspect 22: The apparatus of any one of aspects 18-21, wherein the second side of the at least one compressible pad is planar or generally planar.

Aspect 23: The apparatus any one of aspects 18-22, wherein the at least one compressible pad, in cross sections in planes parallel to the surface of the second side of the compressible pad, has a square, rectangular, or other polygonal outer profile, or a generally square, or generally rectangular, or generally polygonal outer profile.

Aspect 24: The apparatus of any one of aspects 18-23, wherein all of the second portion is positioned peripherally around some or all of the first portion.

Aspect 25: The apparatus of any one of aspects 18-24, wherein the at least one compressible pad comprises a first foam material that defines the first portion and a second foam material that defines the second portion.

Aspect 26: The apparatus of aspect 25, wherein the first foam material has an outer peripheral surface, and wherein the second foam material is coupled to the outer peripheral surface of the first foam material.

Aspect 27: The apparatus of any one of aspects 18-26, wherein the first front-facing surface of the first front-facing side has a convex surface in a direction away from the first back-facing side.

Aspect 28: The apparatus of any one of aspects 18-27, wherein the first front-facing surface of the first front-facing side has a surface shape comprising a partial spherical, ellipsoidal, paraboloidal or hyperboloidal shape.

Aspect 29: The apparatus of any one of aspects 18-28, wherein the first side of the at least one compressible pad has an outer perimeter, and wherein the apex is spaced from the outer perimeter of the first side.

Aspect 30: The apparatus of any one of aspects 18-29, wherein the first side of the at least one compressible pad has an outer perimeter, and wherein the apex is located approximately at the centroid of the area bounded by the outer perimeter.

Aspect 31: The apparatus of any one of aspects 18-30, wherein the at least one compressible pad comprises foam.

Aspect 32: The apparatus of any one of aspects 18-31, wherein the first surface is spaced from the second surface along a first axis to define a thickness of the at least one compressible pad, wherein the thickness of the at least one compressible pad varies across the pad when measured along, or parallel to, the first axis.

Aspect 33: The apparatus of any one of aspects 18-32, wherein the first portion and the second portion are coupled together using one or more patches or strips of hook and loop material on each of the first portion and the second portion.

Aspect 34: The apparatus of any one of aspects 18-33, wherein the at least one compressible pad comprises two compressible pads.

Aspect 35: The apparatus of any one of the preceding aspects, further comprising at least one marking indicating a location for attaching the electrode array to the at least one array attachment element.

Aspect 36: The apparatus of aspect 35, wherein the at least one marking comprises a marking indicating a position for placing a center of the electrode array.

Aspect 37: The apparatus of aspect 35, wherein the at least one marking comprises a marking indicating a position for placing an outer perimeter of the electrode array.

Aspect 38: A method of using the apparatus of any one of the preceding aspects, the method comprising:
securing an electrode array to the at least one array attachment element;
sitting in the seat portion of the chair; and
pressing a back against the electrode array.

Aspect 39: The method of aspect 38, further comprising holding the back against the electrode array for at least 10 seconds.

Aspect 40: A support structure that is configured to be secured to a backrest of a chair, the backrest having a forward-facing side, the support structure comprising:
at least one array attachment element comprising an adhesive or a hook or loop material that is configured to releasably couple to an electrode array,
wherein the support structure is configured to be secured to the backrest of the chair so that the at least one array attachment element is on the forward-facing side of the backrest.

Aspect 41: The support structure of aspect 40, wherein the at least one array attachment element is coupled to the electrode array.

Aspect 42: The support structure of aspect 41 further comprising a removable release liner covering the electrode array.

Aspect 43: The apparatus of aspect 40, wherein the support structure comprises an elastic material that resiliently stretches to receive at least a portion of the backrest.

Aspect 44: The apparatus of aspect 40, wherein the support structure further comprises at least one adjustable strap, wherein the support structure forms a loop comprising the at least one adjustable strap, wherein the adjustable strap is configured to reduce an operative circumference of the loop of the support structure to secure the support structure to the backrest.

Aspect 45: The apparatus of aspect 40, wherein the backrest has a rear-facing side, wherein the support structure comprises:
at least one magnet; and
at least one magnetic material,
wherein the at least one magnet is positioned on one of the forward-facing side or the rear-facing side of the backrest, wherein the at least one magnetic material is positioned on the other of the forward-facing side or the rear-facing side of the back rest, and wherein the at least one magnet and the at least one magnetic material are coupled together through magnetic attraction through the backrest.

Aspect 46: A method comprising:
securing the support structure of any one of aspects 40-45 to the backrest of the chair so that the at least one array attachment element is on the forward-facing side of the backrest.

Aspect 47: The method of aspect 43, further comprising:
releasably coupling the electrode array to the at least one array attachment element.

Aspect 48: The method of aspect 47, further comprising:
leaning back in the chair to adhere the electrode array to a patient's back.

Aspect 49: A method of applying an electrode array to a person's back, the method comprising:
securing an electrode array to at least one array attachment element located on a chair, wherein the chair has:
   a seat portion; and
   a backrest coupled to the seat portion, the backrest having a forward-facing side;
with the person sitting in the seat portion of the chair, pressing the person's back against the electrode array,
wherein the at least one array attachment element is on the forward-facing side of the backrest and the at least one array attachment element comprises an adhesive or a hook or loop material that is configured to releasably couple to the at least one electrode array.

Aspect 50: The method of aspect 49, further comprising holding the back of the person against the electrode array for at least 10 seconds.

Aspect 51: A method comprising:
attaching a support structure to a backrest of a chair, the backrest having a forward-facing side, the support structure comprising:
   at least one array attachment element comprising an adhesive or a hook or loop material that is configured to releasably couple to an electrode array,
wherein, upon attaching the support structure to the backrest of the chair, the support structure is secured to the backrest of the chair so that the at least one array attachment element is on the forward-facing side of the backrest.

Aspect 52: The method of aspect 51, further comprising:
securing an electrode array to the at least one array attachment element;
sitting in the seat portion of the chair; and
pressing a back against the electrode array.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus comprising:
a chair having:
a seat portion; and
a backrest coupled to the seat portion, the backrest having a forward-facing side; and
at least one array attachment element on the forward-facing side of the backrest, the at least one array attachment element comprising an adhesive or a hook or loop material that is configured to releasably couple to at least one electrode array.

2. The apparatus of claim 1, further comprising:
at least one magnet; and
at least one magnetic material,
wherein the backrest has a rear-facing side,
wherein the at least one magnet is positioned on one of the forward-facing side or the rear-facing side of the backrest, wherein the at least one magnetic material is positioned on the other of the forward-facing side or the rear-facing side of the back rest, and wherein the at least one magnet and the at least one magnetic material are coupled together through magnetic attraction through the backrest, and further wherein the at least one array attachment element on the forward-facing side of the backrest is secured to the backrest of the chair by the at least one magnet and the at least one magnetic material.

3. The apparatus of claim 1, wherein the at least one array attachment element is configured to releasably couple to the at least one electrode array with a release force that is less than an adhesive separation force of the at least one electrode array with skin of a patient.

4. The apparatus of claim 1, wherein the at least one array attachment element on the forward-facing side of the backrest comprises at least one compressible pad.

5. The apparatus of claim 4, wherein each compressible pad of the at least one compressible pad has a first front-facing side having a first front-facing surface which extends outwardly in the front-facing direction to form an apex.

6. The apparatus of claim 1, further comprising at least one compressible pad on the forward-facing side of the backrest, wherein each of the at least one compressible pad has a first front-facing side having a first front-facing surface wherein the first front-facing surface has a convex surface in a front-facing direction.

7. The apparatus of claim 5, wherein the first front-facing side of the at least one compressible pad has an outer perimeter, and wherein the apex is located approximately at the centroid of the area bounded by the outer perimeter.

8. The apparatus of claim 1, wherein the apparatus comprises a support structure that is secured to the backrest, wherein the support structure is configured to couple to the at least one array attachment element to thereby couple the at least one array attachment element to the chair.

9. The apparatus of claim 8, wherein the support structure comprises an elastic material that resiliently stretches to receive at least a portion of the backrest.

10. The apparatus of claim 8, wherein the support structure comprises at least one adjustable strap, wherein the support structure forms a loop comprising the at least one adjustable strap, wherein the adjustable strap is configured to reduce an operative circumference of the loop of the support structure to secure the support structure to the backrest.

11. The apparatus of claim 8, wherein the support structure comprises at least one coupling element that is configured to secure the support structure in a configuration in which the support structure forms a loop that surrounds at least a portion of the backrest.

12. The apparatus of claim 8, wherein the backrest has an upper surface, wherein the support structure comprises at least one adjustable strap that is configured to permit adjustment of a vertical position of the at least one array attachment element relative to the upper surface of the backrest.

13. The apparatus of claim 8, wherein the backrest has a rear-facing side, wherein the support structure comprises:
at least one magnet; and
at least one magnetic material,
wherein the at least one magnet is positioned on one of the forward-facing side or the rear-facing side of the backrest, wherein the at least one magnetic material is positioned on the other of the forward-facing side or the rear-facing side of the back rest, and wherein the at least one magnet and the at least one magnetic material are coupled together through magnetic attraction through the backrest.

14. The apparatus of claim 1, wherein the at least one array attachment element on the forward-facing side of the backrest is non-removably secured to the backrest of the chair.

15. A support structure that is configured to be secured to a backrest of a chair, the backrest having a forward-facing side, the support structure comprising:
at least one array attachment element comprising an adhesive or a hook or loop material that is configured to releasably couple to an electrode array,
wherein the support structure is configured to be secured to the backrest of the chair so that the at least one array attachment element is on the forward-facing side of the backrest.

16. The support structure of claim 15, wherein the support structure further comprises an elastic material that resiliently stretches to receive at least a portion of the backrest.

17. The support structure of claim 15, wherein the support structure further comprises at least one adjustable strap, wherein the support structure forms a loop comprising the at least one adjustable strap, and wherein the adjustable strap is configured to reduce an operative circumference of the loop of the support structure to secure the support structure to the backrest.

18. The support structure of claim 15, wherein the backrest has a rear-facing side, wherein the support structure further comprises:
at least one magnet; and
at least one magnetic material,
wherein the at least one magnet is positioned on one of the forward-facing side or the rear-facing side of the backrest, wherein the at least one magnetic material is positioned on the other of the forward-facing side or the rear-facing side of the back rest, and wherein the at least one magnet and the at least one magnetic material are coupled together through magnetic attraction through the backrest.

19. A method of applying an electrode array to a back of a person, the method comprising:
securing an electrode array to at least one array attachment element located on a chair, wherein the chair has:
a seat portion; and
a backrest coupled to the seat portion, the backrest having a forward-facing side;
with the person sitting in the seat portion of the chair, pressing the person's back against the electrode array,
wherein the at least one array attachment element is on the forward-facing side of the backrest and the at least one array attachment element comprises an adhesive or a hook or loop material that is configured to releasably couple to the at least one electrode array.

20. The method of claim 19, further comprising holding the back of the person against the electrode array for at least 10 seconds.
